(19) United States
(12) Patent Application Publication
(10) Pub. No.: US 2020/0340679 A1
(43) Pub. Date: Oct. 29, 2020

(54) OZONE INJECTION SYSTEM AND METHODS THEREOF FOR INJECTING OZONE INTO A KITCHEN EXHAUST SYSTEM

(71) Applicant: Global Plasma Solutions, Inc., Charlotte, NC (US)

(72) Inventor: Charles Houston Waddell, Roanoke, VA (US)

(21) Appl. No.: 16/857,326

(22) Filed: Apr. 24, 2020

Related U.S. Application Data
(60) Provisional application No. 62/837,754, filed on Apr. 24, 2019.

(51) Int. Cl. F24C 15/20; A61L 2/20; A61L 9/015

(52) U.S. Cl. CPC F24C 15/20; A61L 2/202; A61L 2209/212; A61L 2209/111; A61L 9/015

(57) ABSTRACT

An ozone injection system that includes an ozone generator device with an outlet for dispersing ozone, and a first elongate device with a first end and a second end. The first end of the first elongate device is engaged to the outlet of the ozone generator device. An elbow portion with a first end and a second end, and the first end of the elbow portion is engaged to the second end of the first elongate device. A second elongate device with a first end and a second end, the first end of the second elongate device is engaged to the second end of the elbow portion, and at least one opening is located on the second elongate device.

### CROSS REFERENCE TO RELATED PATENT APPLICATION

The present patent application/patent claims the benefit of priority of co-pending U.S. Provisional Patent Application No. 62/837,754, filed on Apr. 24, 2019, and entitled “OZONE INJECTION SYSTEM AND METHODS THEREOF FOR INJECTING OZONE INTO A KITCHEN EXHAUST SYSTEM,” the contents of which are incorporated in full by reference herein.

### FIELD OF THE INVENTION

The present invention relates generally to a system and method designed to produce ozone, mounted within a practical distance from a cooking hood, that uses a conduit, such as stainless steel or black iron pipe to inject ozone into the hood.

### BACKGROUND OF THE INVENTION

A kitchen exhaust system captures heat and contaminants in the air through initially an exhaust hood. The heat and contaminants are then pulled, by the use of an exhaust fan, through a filter, extraction baffles, and ductwork. The air, also containing odors from the food being cooked below the kitchen exhaust system, releases into the outside air located external a residence or building. The transportation of the air through the filters, baffles, and ductwork, after a while, results in grease buildup on these components, resulting in a higher risk of duct fires and extensive and expensive cleaning bills to remove the grease and other build-up from the ductwork and baffles. Furthermore, the odor eliminated into the outside air, can be problematic if it migrants into surrounding offices, buildings, and apartments.

It is an object of the present invention to provide a system mounted within a kitchen hood for injecting ozone, prior to the filters and baffles, for injecting ozone and treating the air, contaminated with heat and other contaminants, prior to the air proceeding through the filter, baffles, and ductwork. The ozone reduces the grease build-up and reduces odors emanating into the outside air from the kitchen exhaust system.

### BRIEF SUMMARY OF THE INVENTION

According to an embodiment of the present invention, a system to produce ozone, mounted within a practical distance from a cooking hood, that uses a conduit, such as stainless steel or black iron pipe to inject ozone into the hood, prior to any filters.

According to another embodiment of the present invention, the system has an ozone injection system that evenly distributes the ozone across the entire width of the hood.

According to yet another embodiment of the present invention, the system has an ozone injection system that distributes the ozone from a single outlet within the hood.

According to yet another embodiment of the present invention, the system wherein a control signal is sent to the ozone device from the hood system, allowing the ozone output to ramp up and down based on cooking operations or odor and grease load.

According to yet another embodiment of the present invention, the system is interlocked with the hood exhaust fan so the device can only operate when the hood exhaust fan is operating.

According to yet another embodiment of the present invention, the system is designed where the entire device is mounted within the hood prior to the filters and no piping is required.

According to yet another embodiment of the present invention, the system has a control signal is sent to the device from the hood system, allowing the ozone output to ramp up and down based on cooking operations or odor and grease load.

According to yet another embodiment of the present invention, the system is interlocked with the hood exhaust fan so the device can only operate when the hood exhaust fan is operating.

According to yet another embodiment of the present invention, the system has an ozone reducing media or device may be installed in the exhaust airstream to limit the amount of ozone exiting the hood and associated ductwork.

According to yet another embodiment of the present invention, the system may include particle filtration that can be installed in the exhaust airstream to limit residual smoke and particles.

According to yet another embodiment of the present invention, the system wherein an ozone sensor may be installed in the exhaust airstream to control the amount of ozone produced.

According to yet another embodiment of the present invention, the system has an ozone sensor may be installed in the indoor space to sense ozone and shut the ozone generator off when a preset ozone limit has been met.

According to yet another embodiment of the present invention, the system has an ozone sensor that may be installed in the indoor space to sense ozone and shut the ozone generator off when a preset ozone limit has been met.

### BRIEF DESCRIPTION OF THE DRAWINGS

The present invention is illustrated and described herein with reference to the various drawings, in which like reference numbers denote like method steps and/or system components, respectively, and in which:

FIG. 1 is a schematic of the present invention.

### DETAILED DESCRIPTION OF THE INVENTION

The present invention may be understood more readily by reference to the following detailed description of the invention taken in connection with the accompanying drawing FIGURES, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

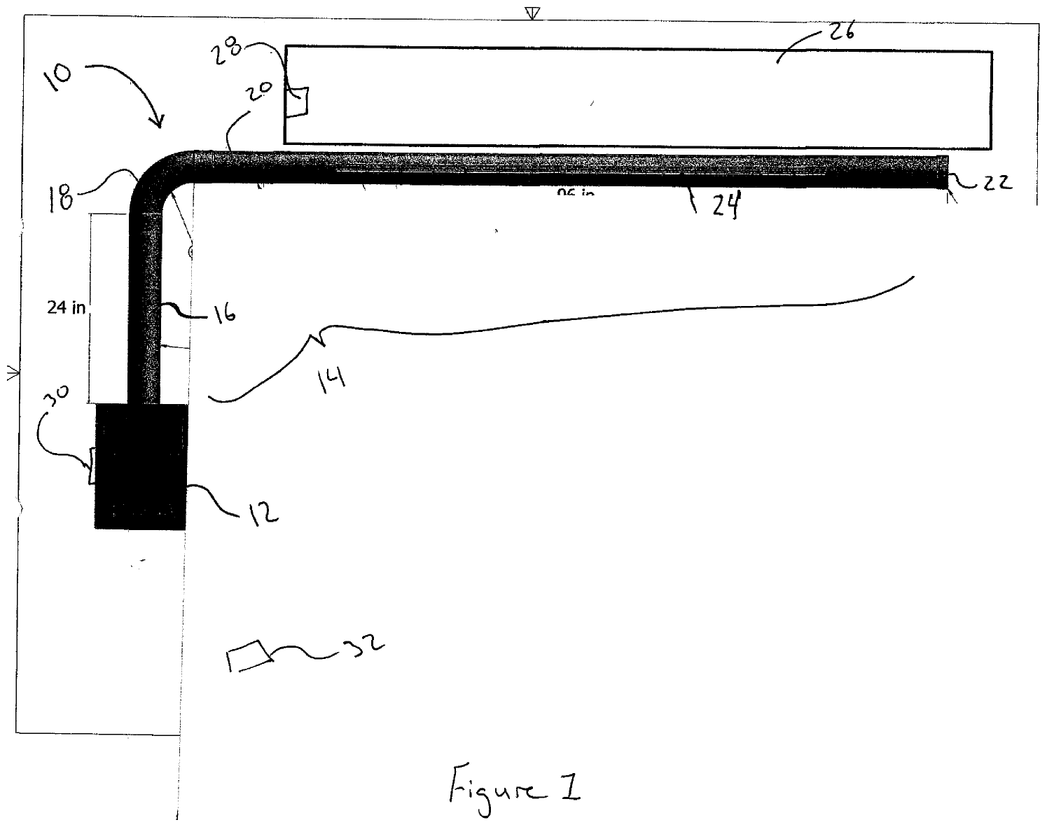

Referring now specifically to the drawings, an ozone injection system is illustrated in FIG. 1 and is shown generally at reference numeral 10. The ozone injection system 10 consists of an ozone generator device 12 engaged to an exhaust assembly 14. The exhaust assembly 14 includes a first elongate device 16, an elbow portion 18, and a second elongate device 20. The ozone generator device 12 preferably uses ceramic plates to generate ozone, instead of glass devices. The ozone generator device 12 may have a stainless steel casing, ceramic ozone generating plates, a voltage indicator, a current (amp) indicator, variable ozone output control, and water washable air inlet filters.

The ozone generator device 12 of the present invention saves energy over traditional carbon filtration systems with no pressure drop creating higher fan energy consumption. The ozone generator device 12 has an outlet for dispersing the ozone generated by the ozone generator device 12. The outlet of the ozone generator device 12 may have a circular cross-section with a diameter between about 1 inch to about 8 inches, more preferably between about 2 inches to about 6 inches, and most preferably from about 3 inches to about 5 inches. The outlet may also have any cross-sectional shape that matches the cross-sectional shape of the first end of the first elongate device 16, allowing the outlet to engaged the first end of the first elongate device 16. The input voltage of the ozone generator device 12 may be 110 volts of alternating current (VAC) or 240 VAC nominal and Amps (A) may be 3.2 A @ 110 Volts(V) or 1.5 A at 240 V. The power may be 350 Watts. A suitable ozone generator device 12 is the KOG-50 that may be purchased from Global Plasma Solutions, Inc. in Charlotte, N.C.

The first elongate device 16 of the exhaust assembly 14 has a first end, a second end, a hollow interior portion, an interior surface around the hollow interior portion, and an exterior surface on the exterior of the first elongate device 16. The first elongate device may be a device with a circular cross-section, as illustrated in FIG. 1. As mentioned above, the first end of the first elongate device 16 is engaged to the outlet of the ozone generator device 12 and extends laterally upward from the top portion of the ozone generator 12. The second end of the first elongate device 16 is engaged to an elbow portion 18. The elbow portion has a first end and a second end. The second end of the first elongate device 16 is engaged to the first end of the elbow portion 18. The elbow portion has a hollow interior portion, an interior surface around the hollow interior portion, and an exterior surface on the exterior of the elbow portion 18. The elbow portion 18 has a longitudinal section and a lateral section joined by a curved element, allowing the ozone to enter the elbow portion through the first end. As the ozone travels through the elbow portion, the flow of the ozone changes almost 90 degrees before exiting the elbow portion through the second end.

The elbow 18 changes the direction of the exhaust assembly 14 from the longitudinal direction to the lateral direction, thus changing the flow of the ozone carried within the exhaust assembly 14. The second end of the elbow 18 is engaged to the first end of a second elongate device 20. The second elongate device 20 of the exhaust assembly 14 has a first end, a second end, a hollow interior portion, an interior surface around the hollow interior portion, and an exterior surface on the exterior of the second elongate device 20. The second elongate device 20 may be a device with a circular cross-section, as illustrated in FIG. 1. As mentioned above, the first end of the second elongate device 20 is engaged to the second end of the elbow portion 18. The second end of the second elongate device 20 is engaged to a cap 22. The cap 22 is secured to the second end of the second elongate device 20 by a fastening device, such as self tapping screws.

The cap 22 is engaged to the second end of the second elongate device 20. At least one opening 24 is disposed on the side of the second elongate device 20. The at least one opening 24 extends from the external surface to the internal surface of the second elongate device 20, allowing the ozone flowing through the hollow interior portion to exit the second elongate device 20 through the opening 24. The second elongate device 20 may have two or more openings 24 disposed on the side of the second elongate device 20. The two or more openings 24 extend from the external surface to the internal surface of the second elongate device 20, allowing the ozone flowing through the hollow interior portion to exit the second elongate device 20 through the two or more openings 24. The second elongate device 20 may have a plurality of openings 24 disposed on the side of the second elongate device 20. The plurality of openings 24 extend from the external surface to the internal surface of the second elongate device 20, allowing the ozone flowing through the hollow interior portion to exit the second elongate device 20 through the plurality of openings 24.

As illustrated, the opening 24 may be an elongated slot. If the second elongate device 20 has two or more openings, the openings are spaced apart from each other. If the elongate device has a plurality of openings 24, the openings 24 may be arranged in two rows—one row above another on the side of the second elongate device 20. The first elongate device 16, elbow 18, and second elongate device 20 may be composed of stainless steel or black iron pipe.

The second elongate device 20 is engaged to the hood 26 by a retention device, such as a pipe clamp and bracket. The first end of the first elongate device 16 is engaged to the outlet of the ozone generator device 12 by an adhesive, mechanical device, such as a clamp, or friction fit, wherein the outlet of the ozone generator device 12 fits within the first end of the first elongate device 16 or the first end of the first elongate device 16 within the outlet of the ozone generator device 12. The second end of the first elongate device 16 is engaged to the first end of the elbow portion 18 by an adhesive, mechanical device, such as a clamp, or friction fit, wherein the second end of the first elongate device 16 fits within the first end of the elbow portion 18 or the second end of the first elongate device 16 within the second end of the elbow portion 18. The second end of the elbow portion 18 is engaged to the first end of the second end of the second elongate device 20 by an adhesive, mechanical device, such as a clamp, or friction fit, wherein the second end of the elbow portion 18 fits within the first end of the second elongate device 20 or the second end of the elbow portion 18 within the first end of the second elongate device 20.

The first elongate device 16 is between about 10 inches to about 35 inches in length, more preferably between about 18 inches to about 30 inches in length, and most preferably between about 20 inches to 28 inches in length. The second elongate device 20 is between about 80 inches to about 120 inches, more preferably between about 90 inches to about 110 inches, and most preferably between about 92 inches to about 100 inches. The diameter of the first elongate device 16, the elbow 18, and the second elongate device 20 is between about 2 inches and about 6 inches, more preferably about 3 inches to about 5 inches, and more preferably about 4 inches. The first elongate device 16 and the and the second elongate device 20 may be cut to fit the length desired by the user.

The ozone injection system 10 is designed to be installed in conjunction with a kitchen exhaust system. The kitchen exhaust system may include a hood 26, a fan system, including a fan, filters, baffles, exhaust ductwork, and a system or device to provide adequate make-up air. The second elongate device 20 is installed to allow the injection of ozone directly into the kitchen hood, prior to the filters and/or baffles. The second elongate device 20 with at least one opening 24 or two or more openings 24 is mounted such that the ozone can be dispersed at a specific location or locations within the hood 26 or hood assembly. The openings 24 may distribute the ozone prior to any filters and/or baffles and/or any other filters that may be installed on the kitchen exhaust system. In an embodiment when the second elongate device 20 has a plurality of openings 24, the second elongate device 20 is preferably mounted such that the openings 24 can evenly distribute ozone across the entire width of the kitchen hood 26. The openings 24 may distribute the ozone prior to any filters and/or baffles and/or any other filters that may be installed on the kitchen exhaust system.

The exhaust hood captures heat and contaminants in the air through the use of filters, extraction baffles, and water mist systems. The most common type of exhaust hood is a large box housing with an open bottom. There are two types of exhaust hoods:

Type 1 hoods are designed to handle grease:

Type 2 hoods are designed to collect steam, vapor, heat and odors—not grease.

The exhaust ductwork transfers the contaminated air, cooking heat, and grease vapors from the hood 26 to the fan. The exhaust ductwork may be contained within an enclosure constructed of gypsum, plaster, concrete, or ceramic tiles for aesthetic appeal. Exhaust fans suck the contaminated air and heat captured in the hood 26 through the filter(s) and baffles and ductwork for extraction out of the housing or building. The hood is installed over a fryer, stove, griddle, or other like cooking surface or device.

The second elongate device 20 may be positioned in such a way that the opening 24 or openings 24 may inject ozone into the hood 26 and prior to any filters within the exhaust system. If the second elongate device 20 has a plurality of openings 24, the openings 24 are spaced along a predetermined length of the second elongate device 20. The predetermined length is a length similar to the length of the opening in the hood 26, or a length similar to the length of the cooking surface and cooking device that is positioned below the hood 26. Such an arrangement allows ozone to be evenly distributed across the entire length of the hood 26 or the area of the hood 26 that would encounter the majority of the grease, steam, heat, vapor, and odors. The length would be the largest distance from one side to the other side of the hood 26, cooking surface, or cooking device. In another embodiment, the entire ozone injection system 10 may be mounted within the hood 26.

The ozone injection system 10 may be operationally coupled and/or communicatively coupled to the fan of the fan system. When the ozone injection system is operationally coupled, the ozone injection system 10 only operates by dispersing ozone when the fan is operating. The fan of the fan system may be operationally coupled to the ozone generator device 12, so that when the fan is turned “on” or operating, the ozone generator device 12 is turned “on” and begins generating and dispersing ozone. Similarly, when the fan of the fan system is turned “off” or ceases operating, the ozone generator device is turned “off” or ceases to produce and distribute ozone. The ozone injection system 10 may be communicatively coupled to the fan of the fan system. When the ozone injection system is communicatively coupled, the ozone injection system senses when the fan is turned “on” or operating, the ozone generator device 12 then turns “on” at a predetermined time after the fan is turned “on” or operating. Similarly, the ozone injection system senses when the fan is turned “of” or ceases operation; therefore, the ozone generator device 12 turns “off” or ceases operating at a predetermined time after the fan is turned “off” or ceases operating.

The ozone injection system may include a sensor 28 that senses cooking operations and/or the odor and grease load in the gas entering the hood 26. The sensor 28 is communicatively coupled to the system 10, and specifically to the ozone generator device 12. The sensor 28 may sense the cooking operation, and based upon a predetermined operation, the sensor 28 sends a signal to a receiver 30 in the ozone generator device 12. Based upon the signal received from the sensor 28, the ozone generator device 12 increases or decreases the amount of ozone produced and distributed. For example, if bacon is being cooked on a griddle that produces large amounts of grease and smoke, compared to the cooking of other items such as eggs, the sensor 28 senses this cooking operation and sends a signal to the receiver 30 of the ozone generator device 12 to increase the production and distribution of ozone. The sensor 28 may also sense the odor or grease load entering the hood 26 and based upon a predetermined amount of odor or grease entering the hood 26, the sensor 28 sends a signal to the receiver 30 in the ozone generator device 12. Based upon the signal received from the sensor 28, the ozone generator device 12 increases or decreases the amount of ozone produced and distributed. For example, if bacon is being cooked on a griddle that produces large amounts of grease, compared to the cooking of other items such as eggs, the sensor 28 senses large amount of grease entering the hood 26 and sends a signal to the receiver 30 of the ozone generator device 12 to increase the production and distribution of ozone. When the bacon is cooked and removed from the griddle and the user begins cooking hash browns, the grease load decreases and the sensor 28 senses the reduced amount of grease entering the hood 26 and sends a signal to the receiver 30 of the ozone generator device 12 to decrease the production and distribution of ozone.

The sensor 28 may also sense the amount of ozone produced and distributed in the exhaust airstream and based upon a predetermined amount of ozone in the airstream, the sensor 28 sends a signal to the receiver 30 in the ozone generator device 12. Based upon the signal received from the sensor 28, the ozone generator device 12 increases or decreases the amount of ozone produced and distributed. A second sensor 32 may be installed in the indoor space near the ozone injection system 10. The second sensor 32 senses the amount of ozone within the indoor space near the ozone injection system 10 and based upon a predetermined amount of ozone in the airstream, the second sensor 32 sends a signal to the receiver 30 in the ozone generator device 12. Based upon the signal received from the sensor 28, the ozone generator device 12 decreases the amount of ozone produced and distributed or may turn “off” or cease operation of the ozone generator device 12.

Additionally, the ozone injection system 10 may include a particle filtration media installed in the exhaust airstream to limit residual smoke and particles. Additionally, the ozone injection system 10 may include a reducing media or an ozone reducing device installed in the exhaust airstream to limit the amount of ozone exiting the hood 26 or the ductwork of the kitchen exhaust system.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention and are intended to be covered by the following claims.

What is claimed is:

1. An ozone injection system, comprising:
an ozone generator device with an outlet for dispersing ozone;
a first elongate device with a first end and a second end, the first end of the first elongate device is engaged to the outlet of the ozone generator device;
an elbow portion with a first end and a second end, the first end of the elbow portion is engaged to the second end of the first elongate device;
a second elongate device with a first end and a second end, the first end of the second elongate device is engaged to the second end of the elbow portion; and
at least one opening is located on the second elongate device.

2. The ozone injection system of claim 1, further comprising a cap engaged to the second end of the second elongate device.

3. The ozone injection system of claim 1, wherein the first elongate device, elbow portion, and second elongate device have a circular cross-section.

4. The ozone injection system of claim 1, wherein the second elongate device includes a plurality of openings.

5. The ozone injection system of claim 1, further comprising a sensor communicatively coupled to the ozone generator device for increasing or decreasing the production and distribution of ozone.

6. An ozone injection system of claim 1, wherein the opening is an elongate slot.

7. An ozone injection system, comprising:
an ozone generator device with an outlet for dispersing ozone, the ozone generator device contains ceramic plates to generate ozone;
a first elongate device with a first end and a second end, the first end of the first elongate device is engaged to the outlet of the ozone generator device;
an elbow portion with a first end and a second end, the first end of the elbow portion is engaged to the second end of the first elongate device;
a second elongate device with a first end and a second end, the first end of the second elongate device is engaged to the second end of the elbow portion; and
at least one opening is located on the second elongate device.

8. The ozone injection system of claim 7, further comprising a cap engaged to the second end of the second elongate device.

9. The ozone injection system of claim 7, wherein the first elongate device, elbow portion, and second elongate device have a circular cross-section.

10. The ozone injection system of claim 7, wherein the second elongate device includes a plurality of openings.

11. The ozone injection system of claim 7, further comprising a sensor communicatively coupled to the ozone generator device for increasing or decreasing the production and distribution of ozone.

12. An ozone injection system of claim 7, wherein the opening is an elongate slot.

13. A method for generating and dispersing ozone, comprising:
providing an ozone injection system including an ozone generator device with an outlet for dispersing ozone, a first elongate device with a first end and a second end, the first end of the first elongate device is engaged to the outlet of the ozone generator device, an elbow portion with a first end and a second end, the first end of the elbow portion is engaged to the second end of the first elongate device, a second elongate device with a first end and a second end, the first end of the second elongate device is engaged to the second end of the elbow portion, and at least one opening is located on the second elongate device;
producing ozone in the ozone generator device,
dispersing ozone through the at least one opening in the second elongate device.

14. The method for generating and dispersing ozone according to claim 13, further comprising a sensor for sensing the odor or grease load.

15. The method for generating and dispersing ozone according to claim 13, further comprising a sensor for sensing a cooking operation.

16. The method for generating and dispersing ozone according to claim 13, wherein the second elongate device is placed within a kitchen exhaust hood.

17. The method for generating and dispersing ozone according to claim 13, further comprising a sensor for sensing ozone in indoor air.
